(19) United States
(12) Patent Application Publication
(10) Pub. No.: US 2006/0074429 A1
(43) Pub. Date: Apr. 6, 2006

(54) Adjustable angle handle for surgical instruments

(76) Inventors: James D. Ralph, Bethlehem, PA (US); Thomas N. Troxell, Pottstown, PA (US); Stephen L. Tater, Montville, NJ (US)

Correspondence Address: NORRIS MCLAUGHLIN & MARCUS, P.A., P O BOX 1018, SOMERVILLE, NJ, 08876, US

(21) Appl. No.: 10/959,572

(22) Filed: Oct. 6, 2004

(51) Int. Cl. A61B 17/16

(52) U.S. Cl. 606/84

(57) ABSTRACT

An adjustable angle handle for a surgical instrument, capable of easy adjustment to a number of angles, permitting the handle of the instrument to be grasped with one hand, and the other hand to guide the operating end of the instrument, said handle permanently affixed to an instrument, or capable if attachment to a number of instruments by means of a firm but releasable fastening to the shaft of the instrument, as with a Hudson fitting; the adjustable angle handle comprising a hand gripping portion with a distally mounted transverse pin with a key, for a keyhole in the proximal end of the instrument, or the instrument retainer shaft.

### FIELD OF THE INVENTION

The present invention relates to surgical instruments and particularly to orthopedic surgical instruments for cutting and scraping bone.

### BACKGROUND OF THE INVENTION

When using certain orthopedic surgical instruments, such as curettes and chisels, for the removal of osteophytes, the surgeon must apply a great a degree of force. Often, this requires the surgeon to use a two handed grip on the straight handle of the instrument. Depending on the angle with which the force may be applied, the two-handed grip may also limit the amount of force that may be applied, and make it difficult to control the operating end of the instrument. In particular, when cleaning out the long bone prior to a hip implant, the surgeon generally grasps the handle of the straight curette with both hands, and moves it in an up-and-down direction. Being able to adjust the angle of the handle with respect to the shaft of the curette will give the surgeon a better angle for cutting, and a better grip, and enable the surgeon to apply force at the handle using only one hand, and use his/her other hand to guide the operating end of the instrument.

### SUMMARY OF THE INVENTION

It is an object of the present invention to provide an easily adjustable, angled handle for surgical instruments, which provides a better grip for the surgeon, for better overall control and manipulation of the instrument.

It is an object of the invention to provide an adjustable angle handle for a surgical instrument which permits the surgeon to control the instrument with one hand, and guide the operating end of the instrument with the other hand.

It is an object of the invention to provide and adjustable angle handle adaptable for use with a number of instruments, to thereby reduce the cost of adjustable angled handles for surgical instruments.

It is an object of the invention to provide an angled handle for a surgical instrument that may be easily adapted for use by both right and left handed surgeons.

It is an object of the invention to provide a multiple user adjustable angle handle for surgical instruments, to reduce the number of handles needed in inventory, and reduce the cost of adjustable angle handles for surgical instruments.

It is another object of the invention to increase the ease of use of various surgical instruments, by permitting the one-handed wielding of the handle; permitting the surgeon a better angle of approach with the instrument. In particular, use in the adjustable angle handle permits easier use of the instrument in the surgical environment regardless of available operating space requirements.

These objects, as well as other objects which, will become apparent from the discussion that follows, are achieved, in accordance with the present invention, which comprises an adjustable angle handle for a surgical instrument, or an adjustable angle handle for a multiple instrument retainer shaft and instrument connector, for use with a number of instruments. The adjustable angle handle has a hand gripping portion rotatably connected to an instrument, or the multiple instrument retainer shaft, with a transverse pin at the distal of the hand gripping portion, passing through a keyhole at the proximal end of the instrument, or multiple instrument retainer shaft. The pin bears a key capable of making a number of fastenings within the keyhole. The pin may be mounted to a housing, fixedly attached to the distal and of the hand gripping portion. A compression spring disposed within the housing may be used to urge the key into the keyhole, fixing the angle of the hand gripping portion with respect to the instrument or the multiple instrument retainer shaft. To easily adjust the angle of the handle, the pin may be extended to form a release pin, opposite the compression spring, so that pressing the release pin overcomes the expansion force of the spring and pushes the key out of the keyhole, permitting free rotation of the hand gripping portion with respect to the instrument or instrument loader shaft until the release pin is released. In its most preferred embodiment, the housing comprises a right retainer housing with an opening for the extended release pin, and a left retainer housing with an opening, such as at 42, to permit the smooth, slideable, non-rotatable movement of the pin, while not engaging the spring.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is an exploded perspective view of a surgical instrument with an adjustable angle handle according to the present invention.

FIG. 2 is an exploded perspective view of an adjustable angle handle for use with one or more surgical instruments, according to the present invention.

FIG. 3 is a side view of the adjustable angle handle of FIG. 2.

FIG. 4 is a cross-sectional view of the adjustable angle handle of FIG. 3, viewed along lines 4-4.

FIG. 5A is a side view of the right retainer housing of FIGS. 1 and 2.

FIG. 5B is a top view, in partial cross-section, of the right retainer housing of FIGS. 1 and 2.

FIG. 5C is a cross-section of the right retainer housing of FIG. 5A, taken along line A-A.

FIG. 6A is a side view of the left retainer housing of FIG. 2.

FIG. 6B is a top view, in partial cross-section, of the left retainer housing of FIG. 2.

FIG. 6C is a cross-section of the left retainer housing of FIG. 6A, taken along line A-A.

FIG. 7 is a top perspective view of the instrument loader shaft of the adjustable angle handle of FIG. 2.

FIG. 8 is a top perspective view of the instrument retaining cap of adjustable angle handle of FIG. 2.

FIG. 9 is a top perspective view of the retainer release housing of adjustable angle handle of FIG. 2.

### DESCRIPTION OF THE PREFERRED EMBODIMENTS

The new handles are especially advantageous for use with orthopedic surgical instruments, such as curettes, chisels, taps and probes; and most especially, with larger chisels. They may also be used with drills.

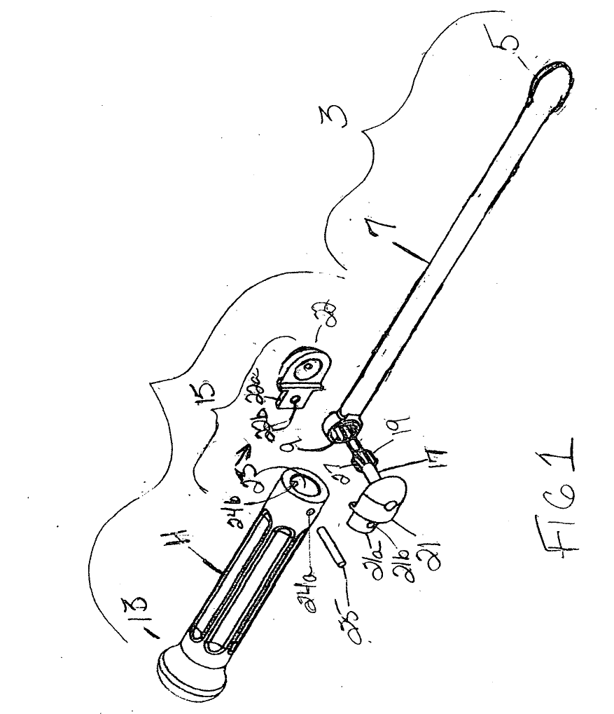

The preferred embodiments of the present invention will now be described with reference to FIGS. 1-9 of the drawings. Identical elements in the various Figures are designated with the same reference numerals.

The exploded perspective view of FIG. 1 illustrates the interconnecting parts of a surgical instrument with an adjustable angle handle according to the present invention. As shown in FIG. 1, the adjustable angle handle, 1, of the surgical instrument is connected to the proximal end of the instrument shaft, with the operating end, 5, of the instrument at the distal end of the shaft, 7. The instrument illustrated in FIG. 1 is a curette however the adjustable angle handle of the present invention is easily adaptable to many forms of surgical instruments, such as chisels, and even drills. As shown in FIG. 1, the shaft, or connector, of the instrument terminates in a keyhole, 9. The keyhole, 9, and the key, 19 (described below), comprise the primary means for fixing the angle of the adjustable angle handle of the present invention.

The adjustable angle handle, 1, comprises a hand gripping portion, 11, having a proximal end, 13, and a distal end, 15. A pin, 17, is attached to the distal end other hand gripping portion, transversely to the general line of the distal end of the hand gripping portion, illustrated by an arrow in FIG. 1. Though the entire hand gripping portion illustrated in FIG. 1 comprises a straight handle, it is within the purview of the present invention that the hand gripping portion being nonlinear, jointed, or curved.

A key, 19, is mounted about the pin, 17, which is disposed through the keyhole, 9. The key is capable of making a number of different fastenings within the keyhole, to achieve different angles of rotation of the handle to the instrument.

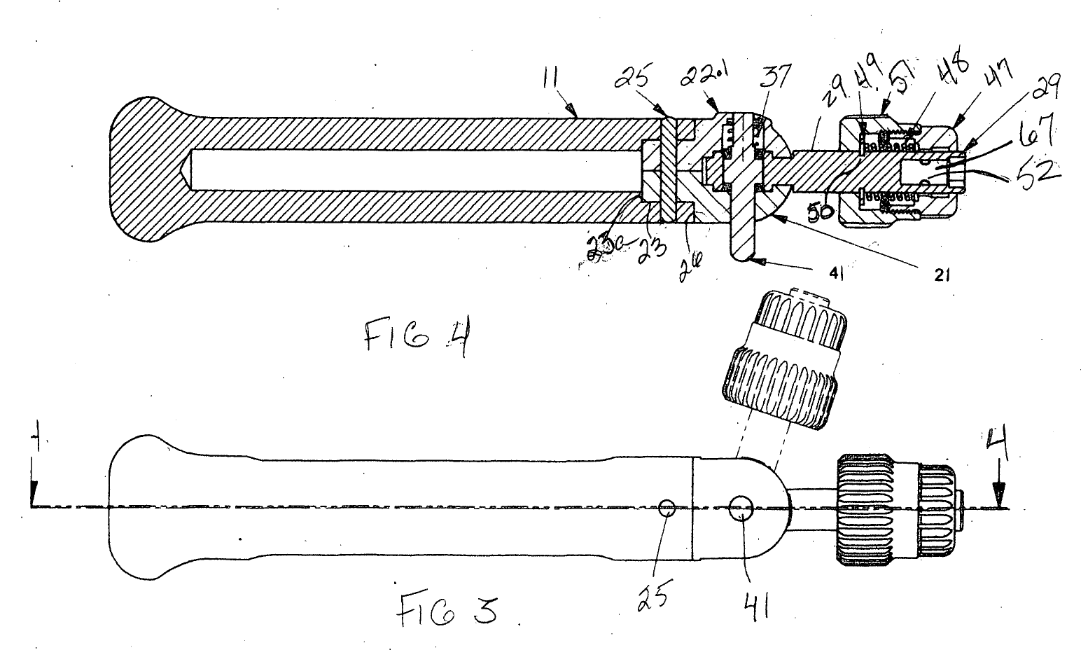

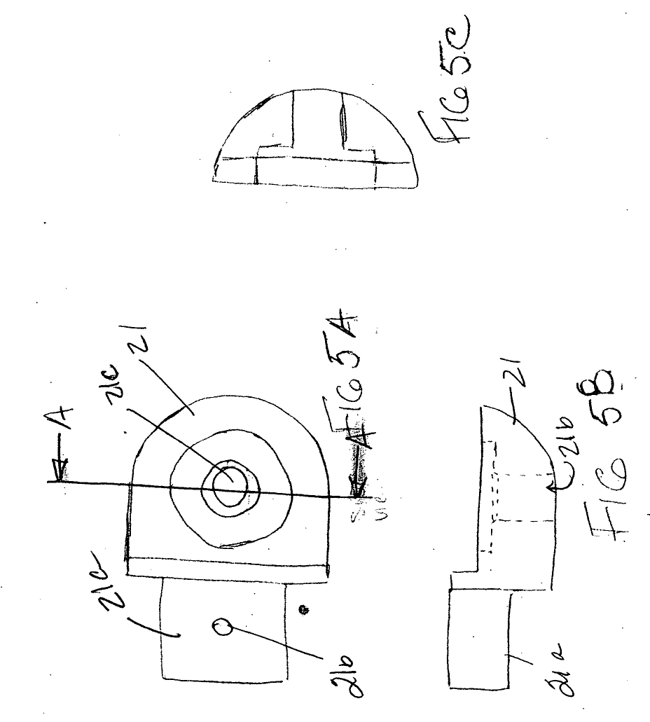

As shown in FIG. 1, the pin, 17, is attached to the distal end of the hand gripping portion of by means of a housing, in this particular embodiment, a two-part housing, designated the right retainer housing, 21, and the left retainer housing, 22. The right retainer housing, illustrated more fully in FIGS. 5A-5C, has a generally half-dome shape with a flange, 21a, at the base of the dome, and an opening 21b, through the flange. Similarly, the left retainer housing has a generally half-dome shape with a flange, 22a, at the base of he half-dome, and an opening 22b, through the flange. In this embodiment the right retainer housing is identical to left retainer housing.

The half-dome shape not only provides easy of assembly, but, in use, provides a “clean sweep”, avoiding unnecessary contact with corners or protrusions, which can lead to unintended repositioning of the instrument, or tears in surgical gloves. In addition, the half-dome shape minimizes the profile of the instrument, giving the surgeon maximum field of vision.

In assembling the surgical instrument of FIG. 1, the pin, 17, with key, 19, is disposed through the keyhole, 9, and mounted within the housing, by bringing together the right and left retainer housing portions, which aligns their retaining openings 21b and 22b. The aligned flanges are then disposed within the bore, 23, of the distal end of the hand gripping portion, and a dowel pin, 25, placed through holes, 24a, 21b, 22b, and 24b, to secure the housing to the distal end of the hand gripping portion. In this, the simplest form of the invention, the angle of the handle may be adjusted by removing the dowel pin, opening up the housing, sliding the key from the keyhole, rotating the shaft with respect to the hand gripping portion, and re-assembling a housing, hand gripping portion, and dowel pin. However, an easier method of adjusting the angle is discussed below in relation to FIG. 2. In the embodiment shown in FIG. 1, the key is a splined key, comprising a number of generally parallel splines, 27, oriented along the length of the pin. Any number of known key and keyhole designs may be easily adapted for use with the present invention.

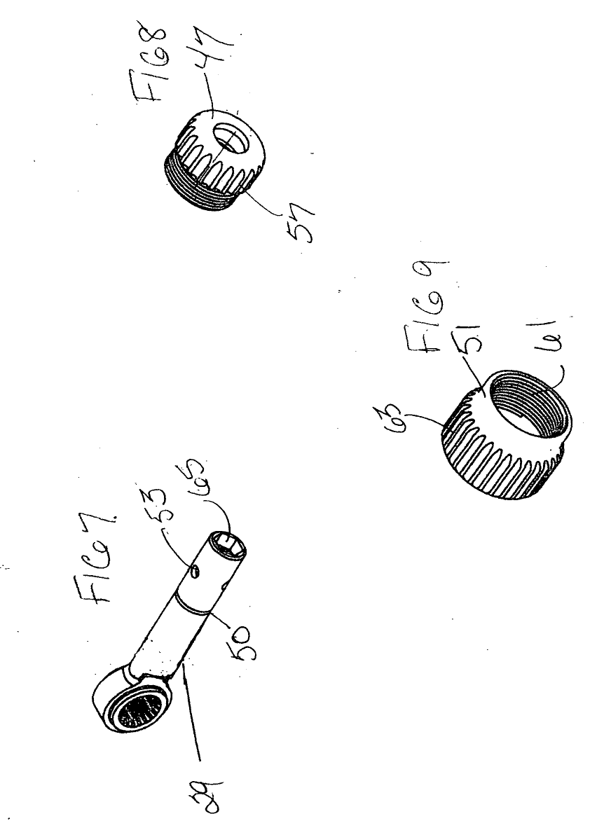

FIG. 2 is an exploded perspective view of an adjustable angle handle according to the present invention which may be used with a number of surgical instruments, and is easily adjusted, without disassembling the parts, by means of a release pin, 41. in this embodiment of the invention, the shaft of the instrument has been replaced by an instrument retainer shaft, 29, an enlarged perspective view of which is shown in FIG. 7. At the distal end, 31, of the instrument retainer shaft is an instrument connector, 33, comprising a quick connect, and/or quick disconnect assembly, as known in the art. In this embodiment the connector is the traditional Hudson fitting, however other known fittings, such as, i.e. a taper lock, a three to four finger collet, or a set screw, may be used to create the instrument connector of the adjustable angle handle of the present invention.

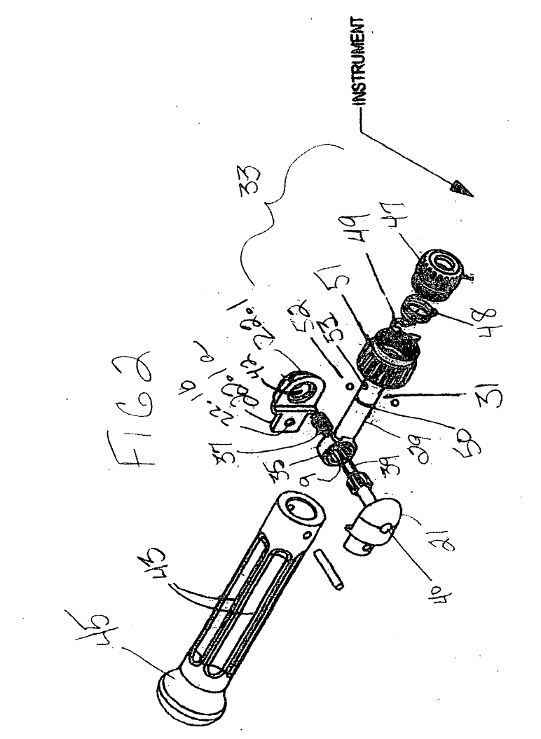

The adjustable angle rotatable connection of the handle illustrated in FIG. 2 includes a keyhole at the proximal end of the instrument retaining shaft; an extended pin, forming a release pin, 41, and a compression spring, 37 disposed within the housing, urging the key into the keyhole. The assembly and operation of the handle are readily described in relation to FIGS. 3 and 4.

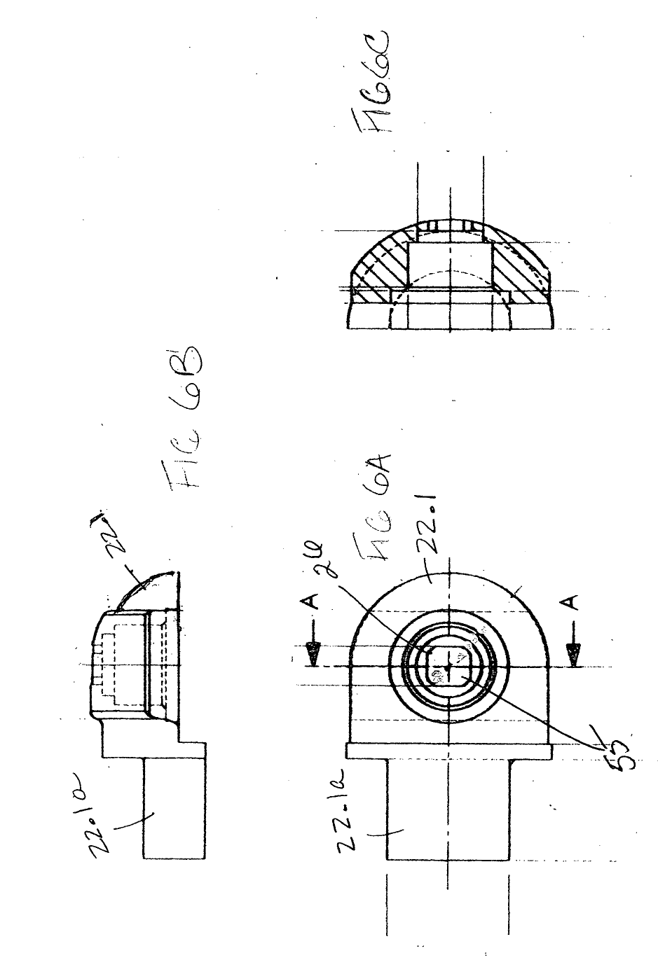

FIG. 3 illustrates a side view of the handle shown in FIG. 2, with an adjustable angle achieved therewith shown in phantom. FIG. 4 is a cross-sectional view of the handle of FIG. 3, taken along the lines 4-4. As may be easily seen in FIG. 4, the right retainer housing, 21, [illustrated more fully in FIGS. 5A-5C,] is not identical to the left retainer housing, 22.1, which is illustrated more fully in FIGS. 6A-6C. The opening, 40, for the pin in the right housing accommodates the slideably mounted, extended pin, 17, creating a release pin, 41. The opening, 42, in the left housing accommodates the end of the spring retaining portion of the pin which has a facetted profile, to provide a slideable, but not rotatable fastening through the opening, 42, in the housing. Prior to assembling the housing, the key, 19 is disposed through the keyhole, 9, and a compression spring, 37 is placed on the spring retaining portion of the pin, 39. The right and left retaining housing portions are then assembled about the extended pin, spring, key, and keyhole.

The flanges and flange openings are aligned and are then inserted into the bore, 23, of the hand gripping portion. As shown, the end of the flanges abut the shoulder, 23a, of the bore, and then shoulder 26, of the flange abuts the end surface of the distal end of the hand gripping portion to stabilize the housing within the hand gripping portion. In this particular embodiment, the extended pin passes through opening 21c, illustrated in FIG. 5A, to the outside of the housing. The other end of the pin is disposed through the squared opening, 55 of the left retainer housing, illustrated in FIG. 6A. To release the key from the keyhole, the extended release pin is pushed toward the housing, overcoming the force of the compression spraying, 37, to dislodge the key from the keyhole.

As may be seen more completely with regard to FIG. 4, the surgeon may wrap his/her hand about the hand gripping portion, 11, and retain the release pin, 41, against the right retainer housing using their thumb. The other hand may then grasp the instrument loader shaft, or the instrument loaded therein, and adjust the angle of the handle. When the desired angle is achieved, the thumb can be released from the release pin, and compression spring will move the key back into the keyhole. Depending on the structure of the key and the keyhole, the handle may be adjusted to a number of different angles.

As illustrated in FIG. 2, the hand gripping portion may also be provided with a molded region to create a gripping surface, 43, which allows the surgeon to more easily grasp and manipulate the handle with one hand. The hand gripping portion may also be provided with an enlarged proximal end stop, 45, to insure the surgeons hand does not slip from the handle, especially when using a one-handed grip.

The instrument connector, 33, on the instrument loader shaft, 29, comprises an instrument retaining cap, 47, a compression spring, 48, an E-ring, 49, in transverse groove, 50, retainer release housing, 51, and instrument retaining balls (at least 3), 52, staked into openings, 53, (at least 3) in instrument retainer shaft 29. Instrument retaining cap, 47 is illustrated more fully in FIG. 8, showing its outer knurled surface, 57, and external threads, 59. The retainer release housing, 51 is more fully illustrated in FIG. 9, showing its internal threads, 61, and outer knurled surface 63.

As shown in FIG. 4, when assembled on the instrument retainer shaft, 29 (clearly illustrated in FIG. 7) the retainer release housing is mounted on the instrument loader shaft, the E-ring is then disposed within the groove, 50 of the instrument retainer shaft, the compression spring, 48 is placed about the instrument retainer shaft, the instrument retaining cap disposed on the instrument retainer shaft, and the exterior threads of the instrument retaining cap secured to the internal threads, 61 of the retainer release housing, 51. To securing an instrument in the instrument retainer shaft, the user pulls back on the retainer release housing, 51, and inserts the instrument shaft, or connector into guide, 61, to align the instrument in the instrument retaining chamber, 67. The guide, 61, illustrated in FIG. 7, is a hexagonal guide, however, it is only necessary that the guide co-operate with the surface(s) of the instrument to orient the instrument in the chamber, such that a tight grip is achieved with the particular instrument connector. Upon release of the retainer release housing, 51, the compression spring pushes it, and the instrument retaining cap forward, and drives the instrument retaining balls down into the groove on the instrument, firmly securing the instrument to the handle.

If desired, the instrument shaft, or connector, may have a shoulder which mates with the guide. When the instrument shaft or connector is in the chamber, the instrument retaining cap, 47, is screwed into the retainer release housing, 51, to firmly grip the shaft of the instrument. To release the instrument, retainer release housing is rotated with respect to the instrument retaining cap, releasing the threads, and compression spring, 48, pushes the instrument retaining cap forward to release the pressure on the shaft of the instrument.

There has thus been shown and described a novel articulating handle for surgical instruments which fulfills all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

I/We claim:

1. A surgical instrument with a distal operating end disposed on a shaft, and an adjustable angle handle fixed to the proximal end of the shaft, said handle comprising,
(a) a hand gripping portion, having a proximal end, and a distal end, a pin attached transversely to the hand gripping portion, at the distal end, and a key mounted on the pin, and
(b) an instrument loader shaft having a distal end attached to the shaft of the instrument, and a proximal end comprising a keyhole rotatably mounted about said pin, said key capable of multiple fastenings within the keyhole, to adjust the angle of rotation of the hand gripping portion to the “instrument loading portion.

2. A surgical instrument as in claim 1, wherein the pin is mounted to a housing attached to the hand gripping portion, and said means for fixing the angle of rotation further comprises a spring retained between the key and the housing, forcing the key into the keyhole.

3. A surgical instrument as in claim 1, wherein the key comprises a splined key, wherein the splines are disposed along the length of the pin.

4. A surgical instrument as in claim 3, further comprising means for releasing the key from the keyhole, comprising an extended pin, slideably mounted to the housing, and extending outside of the housing, opposite the spring, such that pushing the extended pin compresses the spring and forces the key out of the keyhole to permit adjustment of the angle of rotation of the handle.

5. A surgical instrument as in claim 4, wherein the housing comprises mating right and left, half retainer housings, assembled about, and containing, the pin, the key and the keyhole.

6. A surgical instrument as in claim 5, wherein each housing comprises a half-dome shape, with a recessed flange attached to the base thereof, said mating flanges disposed within a bore at the distal end of the hand gripping portion and fixedly retained therein.

7. A surgical instrument as in claim 6, wherein the flanges of the mating housing portions are fixedly retained therein by a dowel pin disposed through opposed holes in the distal end of the hand gripping portion, and mating retaining openings in the flanges.

8. A surgical instrument as in claim 7, wherein the flange further comprises a shoulder adjacent the half-dome, said shoulder extending at an angle to the axis of the bore, and the distal end of the hand gripping portion comprises an end surface mating to the flange shoulder, to stabilize the attachment of the hand gripping portion to the shaft of the instrument.

9. A surgical instrument as in claim 1, wherein the hand gripping portion has an enlarged proximal end stop, and the surface of the hand gripping portion comprises molded regions creating a gripping surface.

10. An adjustable angle handle for surgical instruments having a operating end disposed on the distal end of a shaft, and groove on the proximal end of the instrument shaft, said adjustable angle handle comprising,
a hand gripping portion, having a proximal end, and a distal end, a pin attached at the distal end, transversely to the hand gripping portion, with a key mounted on the pin, and
an instrument retainer shaft, having a distal end with an instrument connector for attachment to the instrument shaft at the groove, and a proximal end comprising a rotatably mounted about said pin, and capable of making a number of fastenings to the key, to adjust the angle of rotation of the hand gripping portion to the instrument retainer shaft.

11. A surgical instrument as in claim 10, wherein the pin is mounted to a housing attached to the hand gripping portion, and said means for fixing the angle of rotation further comprises a spring retained between the key and the housing, forcing the key into the keyhole.

12. An adjustable angle handle for surgical instruments as in claim 10, wherein the key is a splined key, with the splines disposed along the length of the pin.

13. A surgical instrument as in claim 12, further comprising means for releasing the key from the keyhole, comprising an extended pin, slideably mounted to the housing, and extending outside of the housing, opposite the spring, such than pushing the extended pin compresses the spring and forces the key out of the keyhole.

14. A surgical instrument as in claim 13, wherein the housing comprises mating right and left, half retainer housings, assembled about, and containing, the pin, the key and the keyhole.

15. A surgical instrument as in claim 14, wherein each housing comprises a half-dome shape, with a recessed flange attached to the base thereof, said mating flanges disposed within a bore at the distal end of the hand gripping portion and fixedly retained therein.

16. A surgical instrument as in claim 15, wherein the flanges of the mating housing portions are fixedly retained therein by a dowel pin disposed through opposed holes in the distal end of the hand gripping portion, and mating retaining openings in the flanges.

17. A surgical instrument as in claim 15, wherein the flange further comprises a shoulder adjacent the half-dome, said shoulder extending at an angle to the axis of the bore, and the distal end of the hand gripping portion comprises an end surface mating to the flange shoulder, to stabilize the attachment of the hand gripping portion to the shaft of the instrument.

18. A surgical instrument as in claim 10, wherein the hand gripping portion has an enlarged proximal end stop, and the surface of the hand gripping portion comprises molded regions creating a gripping surface.

19. A surgical instrument as in claim 10, wherein the instrument connector comprises a Hudson fitting.

20. A surgical instrument as in claim 13, wherein the instrument connector comprises a Hudson fitting.

21. A surgical instrument as in claim 13 wherein said slideable mounting comprises an opening in left retainer housing for receiving the end of the spring retaining portion of the pin.

22. A surgical instrument as in claim 21, wherein the end of the spring retaining portion of the pin is facetted and the opening in the housing accommodated the facetted pin portion such as to provide a smooth, slideable, but non-rotatable attachment.
